(19) United States
(12) Patent Application Publication
(10) Pub. No.: US 2024/0049988 A1
(43) Pub. Date: Feb. 15, 2024

(54) Kinematics Tracking System And Method

(71) Applicant: Orthosensor, Inc., Dania Beach, FL (US)

(72) Inventors: Benjamin Clarke, Guildford (GB); Andrew Meyer, Plantation, FL (US); Martin Roche, Fort Lauderdale, FL (US); Matthias Verstraete, Chaam (NL)

(73) Assignee: Orthosensor, Inc., Dania Beach, FL (US)

(21) Appl. No.: 18/258,020

(22) PCT Filed: Dec. 27, 2021
(86) PCT No.: PCT/IB2021/062355

§ 371 Date: Jun. 16, 2023

Related U.S. Application Data
(60) Provisional application No. 63/130,455, filed on Dec. 24, 2020.

(51) Int. Cl. A61B 5/11; A61B 5/00

(52) U.S. Cl. CPC A61B 5/112; A61B 5/1122; A61B 5/6828; A61B 5/742; A61B 5/1126; A61B 2562/0219; A61B 2560/0223

(57) ABSTRACT

A kinematics tracking system is described. The kinematics tracking system includes a first device configured to couple to a first segment of a musculoskeletal system and a second device configured to couple to a second segment of the musculoskeletal system. The kinematics tracking system further includes a computer configured to receive measurement data from the first device and the second device. The first device and the second device each have at least one inertial measurement unit (IMU) configured to measure orientation. The computer includes an application configured to support a registration process for the first and second devices. The application is configured to guide a user through at least one movement during the registration process.

### CROSS-REFERENCE TO RELATED APPLICATION(S)

This application claims priority to and all the advantages of U.S. Provisional Patent Application No. 63/130,455, filed on Dec. 24, 2020, the contents of which are incorporated herein by reference.

### FIELD

The present invention pertains generally to measurement of physical parameters, and particularly to, but not exclusively to, measuring orthopedic alignment.

### BACKGROUND

Neural musculoskeletal disorders, such as arthritis or cerebral palsy, are a common cause of disability. These disorders limit people's mobility through the muscle or skeletal pain in the case of osteoarthritis, or by inhibiting to muscle control in the case of cerebral palsy. Many interventions have been developed to improve the mobility of patients with musculoskeletal disorders, and corresponding motion measurement technologies have been invented to evaluate the effectiveness of these treatments and guide the patients through their recovery.

In addition, musculoskeletal trauma or injuries potentially associated with ligamentous failure(s) can be debilitating. These injuries can be treated using reconstruction surgery and/or extensive recovery programs involving physiotherapy. Motion tracking systems can be relevant at that point to guide patients through proper recovery and provide (remote) insight in their recovery by the care team.

In sports, a detailed understanding of the (joint-specific) training load and proper execution of movements can help prevent injuries, avoid over-training while also helping the recovery after an injury and potentially improving the athlete's overall performance. As a result, detailed monitoring of the joints' kinematics is essential to quantify the joint movement and understand the stress that the joint experiences.

### SUMMARY

This section provides a general summary of the disclosure, and is not a comprehensive disclosure of its full scope or all of its features.

In a feature, a kinematics tracking system is disclosed. The kinematics tracking system includes a first device that is configured to measure a first orientation. The first device is coupled to a first segment of a musculoskeletal system of a person. The kinematics tracking system also includes a second device that is configured to measure a second orientation. The second device is coupled to a second segment of the musculoskeletal system of the person. The kinematics tracking system also includes a computer that is configured to receive measurement data from the first device and the second device. The computer includes an application configured to direct the person to perform a registration process. A zero level is determined by kinematic axes coupling rotational joints to the first and second segments. At least one movement including the first segment configured to rotate and the second segment configured to move in a straight line while remaining rotationally free. The measurement data collected during the at least one movement is used to determine a rotation of 6F and 6T of the first device and the second device such that the zero level corresponds to a kinematic axis connecting the rotational joints with a shared joint coupled between the first and second segments.

In another feature, a kinematics tracking system is disclosed. The kinematics tracking system includes a first device that is configured to measure a first motion, a first position, or a first orientation. The first device is coupled to a first segment of a musculoskeletal system of a person. The kinematics tracking system also includes a second device that is configured to measure a second motion, a second position, or a second orientation. The second device being coupled to a second segment of the musculoskeletal system of the person. A computer is configured to receive measurement data from the first device and the second device. The computer includes an application configured to direct the person to perform a registration process comprising a movement. The first segment is configured to rotate and the second segment is configured to move in a straight line while remaining rotationally free. The computer reports on a display when the second segment has moved a predetermined distance or angle.

### BRIEF DESCRIPTION OF THE DRAWINGS

Various features of the system are set forth with particularity in the appended claims. The embodiments herein, can be understood by reference to the following description, taken in conjunction with the accompanying drawings, in which:

FIG. 1 is an illustration of a kinematics tracking system in accordance with an example embodiment;

FIG. 2A is an illustration of a first movement for the registration process in accordance with an example embodiment;

FIG. 2B is an illustration of a second movement for the registration process in accordance with an example embodiment;

FIG. 2C is an illustration of a third movement for the registration process in accordance with an example embodiment;

FIG. 2D is an illustration of a fourth movement for the registration process in accordance with an example embodiment;

FIG. 2E is an illustration of a fifth movement for the registration process in accordance with an example embodiment;

FIG. 3 is a geometric representation of body segments for the first movement in accordance with an example embodiment;

FIG. 4 is a geometric representation of body segments for the second movement in accordance with an example embodiment;

FIG. 5 is a flow chart of a registration process in accordance with an example embodiment;

FIG. 6 block diagram showing an overview of a data flow the registration process in accordance with an example embodiment; and

FIG. 7 is a block diagram showing one or more images of an application for performing the registration process on a computer in accordance with an example embodiment.

### DETAILED DESCRIPTION

Embodiments of the invention are broadly directed to measurement of physical parameters, and more particularly, to a system that supports accurate measurement, improves surgical outcomes, reduces cost, reduces time in surgery, improves recovery after surgery, and reduces risk of injury.

The following description of exemplary embodiment(s) is merely illustrative in nature and is in no way intended to limit the invention, its application, or uses.

Processes, techniques, apparatus, and materials as known by one of ordinary skill in the art may not be discussed in detail but are intended to be part of the enabling description where appropriate. For example, specific computer code may not be listed for achieving each of the steps discussed, however one of ordinary skill would be able, without undo experimentation, to write such code given the enabling disclosure herein. Such code is intended to fall within the scope of at least one exemplary embodiment.

In all of the examples illustrated and discussed herein, any specific materials, such as temperatures, times, energies, and material properties for process steps or specific structure implementations should be interpreted to be illustrative only and non-limiting. Processes, techniques, apparatus, and materials as known by one of ordinary skill in the art may not be discussed in detail but are intended to be part of an enabling description where appropriate. It should also be noted that the word “coupled” used herein implies that elements may be directly coupled together or may be coupled through one or more intervening elements.

Additionally, the sizes of structures used in exemplary embodiments are not limited by any discussion herein (e.g., the sizes of structures can be macro (centimeter, meter, and larger sizes), micro (micrometer), and nanometer size and smaller).

Notice that similar reference numerals and letters refer to similar items in the following figures, and thus once an item is defined in one figure, it may not be discussed or further defined in the following figures.

When measuring joint kinematics, an important consideration is the zero level of each joint. For consistency, joint angles are measured with respect to a pose referred to as “the anatomical position.” In the anatomical position, a person stands straight or lies down flat so that the spine, legs, arms, neck, and head are approximately parallel. The feet point anteriorly, and the palms point anteriorly. The bulk of medical knowledge of human joint kinematics is measured relative to the anatomical position, so devices for measuring joint motion should report angles according to this convention.

One device commonly used to measure musculoskeletal motion is the inertial measurement unit (IMU). IMU sensors measure linear accelerations and angular velocities. When attached to segments of the human body, these sensors can be used to calculate segment linear motion and angular orientation. When several body segments are recorded simultaneously, the angles of the joints coupling two segments can be calculated.

One challenge of calculating joint angles with IMUS is determining the orientation of the sensor on each segment relative to the mechanical axis of the segment. Many methods exist for determining these orientations however defining the zero angle of the measured joints to match the anatomical position remains a challenge. In order to determine the zero angle of the joint, some of these calibration methods require people to orient their body segments at known static positions, such as full joint extension. However, these positions may vary from subject to subject, over the recovery pathway and/or may be unobtainable for subjects with joint mobility disorders. Other zero level calculations require measurement of these joint angles with a goniometer or other motion capture technologies which may be unavailable in some situations and are inevitably prone to measurement errors as these often misidentify the kinematic joint center. Joint angle zero level calculation methods that do not require people to obtain a specific pose, and do not use external measurement devices, would improve the utility of IMUS when measuring joint angles in people with impaired joint mobility. For example, the registration process can be performed in a remote setting where the patient is not with the healthcare provider.

Disclosed herein is a system and method to determine the alignment of inertial measurement unit sensors placed in any location or orientation on two adjacent body segments on the limb of a mammal, in particular the thigh and shank of a human. In one embodiment, body segments correspond to bones of the musculoskeletal system. These inertial measurement units measure tri-axial linear accelerations and tri-axial angular velocities of each attached segment. Using the direction of gravity and Kalman filtering algorithms or the complementary filter, the sensor orientations relative to a fixed coordinate system can be determined.

This invention is not limited to IMU sensors but refers to all sensor modalities that allow the measurement of the orientation of a sensor system, these include but are not limited to the IMU and can include sensors such as, hall effect sensors, marker-based tracking systems, acoustical ranging devices, optical devices, magnetometers, inertial devices, MEMS devices, GPS, inclinometers, and infrared sensors. When these sensors are attached to a body segment, the principal axes of the sensor system are in its general format not aligned with the anatomical axes of the associated segment. As a result, the presented method supports the determination of the offset angles between the sensor system and the anatomical structure. The attachment method of the sensors to the body segment can thereby take various forms, including but not limited to skin-worn patches, screws directly attached to the bones, sensor systems being either subcutaneous or external, or an external coupled navigation system. In one embodiment, the devices coupled to a body segment will include a device configured to measure orientation.

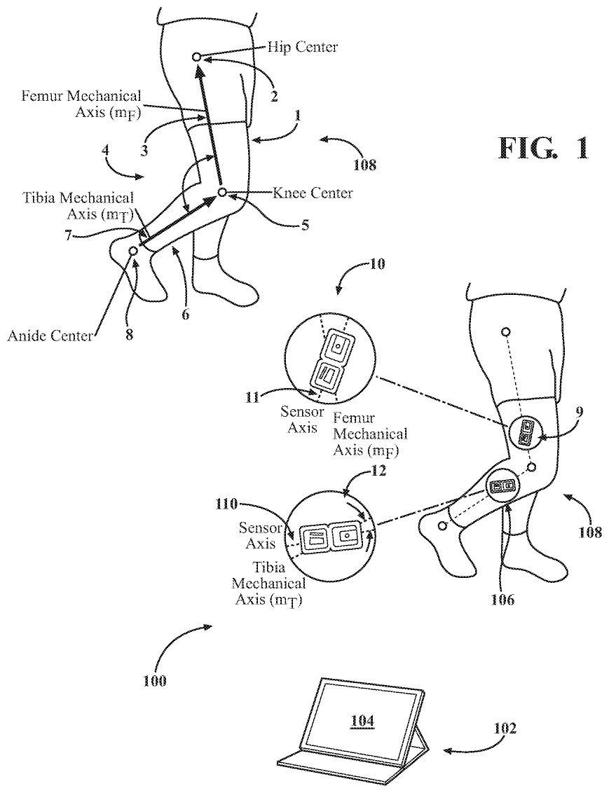

FIG. 1 is an illustration of a kinematics tracking system 100 in accordance with an example embodiment. Kinematics tracking system 100 comprises a device 9, a device 106, and a computer 102 having a display 104. Device 9 and device 106 are configured to measure orientation. In one embodiment, device 9 or device 106 includes electronic circuitry and at least one inertial measurement unit (IMU) for measuring orientation. The electronic circuitry couples to the at least one IMU and is configured to control a measurement process and transmit measurement data to computer 102. In one embodiment, device 9 or device 106 includes a power source, energy harvesting, or is configured to receive power from an external source. In one embodiment, the electronic circuitry can include power management circuitry configured to receive energy by inductive coupling, light coupling, or radio frequency coupling that is harvested and stored in device 9 or device 106 until sufficient energy is stored to power device 9 or device 106 to complete a measurement task. Computer 102 includes one or more software programs for processing measurement data received from device 9 and device 106. Computer 106 can be any device having a processor, digital logic, microprocessor, microcontroller or digital signal processor that can be configured to support the software to process measurement data. For example, computer 102 can be a medical device, a phone, a tablet, a notebook computer, a personal computer, or a hand held device to name but a few. In one embodiment, computer 102 has an application or an “app” that is configured to direct a person through one or more movements to complete the process of registration for device 9 and device 106. In one embodiment, computer 102 includes visual, audible, or haptic feedback related to the registration process. In one embodiment, display 104 can provide visual feedback to support the person in real-time to complete the registration process. This can include instructions on how to perform the registration process as well as real-time feedback as the person performs the registration process.

A leg 108 is illustrated in FIG. 1 showing a hip joint 2, a knee joint 5, and an ankle joint 8 respectively having a hip center, a knee center, and an ankle center of each joint. A musculoskeletal system of leg 108 comprises a femur 1 and a tibia 6. A mechanical axis mF 3 of femur 1 is a straight line from the knee joint center of knee joint 5 to the hip center of hip joint 2. Similarly, a mechanical axis m_{T} 7 of tibia 6 is a straight line from the knee center knee joint 5 to the ankle center of ankle joint 8. A knee angle 4 corresponds to the angle formed between mechanical axis m f 3 and mechanical axis m_{T} 7.

Device 9 of kinematics tracking system 100 is coupled to a first segment of the musculoskeletal system and device 106 of kinematics tracking system 100 is coupled to a second segment of the musculoskeletal system. In the example, device 9 is coupled to a femur of 1 and device 106 is coupled to a tibia 6 of leg 108. Note that device 9 moves with the femur and device 106 moves with the tibia. Since the orientation of device 9 and device 106 relative to a common fixed reference frame (earth, gravity) is known, the angle of the joint coupling the two segments, in FIG. 1 the knee angle 4, can be determined. However, the absolute zero level of knee angle 4 is uncertain since the orientation of device 9 relative to mechanical axis mF 3 of femur 1 and the orientation of device 106 relative mechanical axis m_{T} 7 of tibia 6 are both unknown. Moreover, the zero level of knee angle 4 will vary depending on the way device 9 and device 106 are respectively coupled to femur 1 and tibia 6 of leg 108. This is reflected by an offset angle for device 9 and device 106 respectively δ_{F} 10 and δ_{T} 12 for femur 1 and tibia 6. In biomechanical and clinical research these are typically defined at a known, static calibration pose. The zero level thereby occurs when a person stands in a neutral anatomical position. For the knee of leg 108, this occurs at full extension, when mechanical axis mF 3 of femur 1 and mechanical axis m_{T} 7 of tibia 6 are aligned or parallel. However, determining the zero level of the knee angle 4 can be difficult if not impossible in mobility impaired persons, where a fully extended joint might not be achievable.

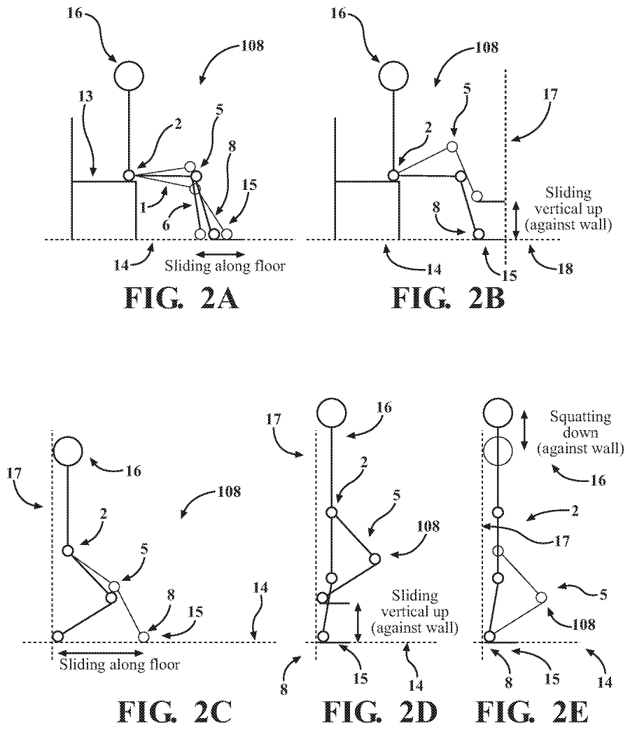

FIG. 2A-E shows a series of activities that supports registration of devices 9 and 106 in accordance with an example embodiment. In the example, the series of activities are used in isolation or combination to determine the rotation 6F 10 and 6T 12 respectively of device 9 and device 106 such that the zero level of the joint, in this case knee joint 5, will reflect a zero level when leg 108 is in full extension, in line with the clinical/biomechanical definition. In one embodiment, the joint participating in each of these activities disclosed herein below will have a first neighboring joint that rotates about a single axis, but does not translate, while a second neighboring joint moves in a straight line along a flat surface. In the example of knee joint 5, neighboring joints comprise hip joint 2 and an ankle joint 8. In the example movements, hip joint 2 will rotate about a single axis but will have no translational motion. Ankle joint 8 moves in a straight line along a flat surface. In one embodiment, the movement occurs over a predetermined distance. In one embodiment, a person 16 seeks to keep the entire movement in one plane. For example, during the activities drawn here, minimal changes to the abduction-adduction and internal-external rotation of the hip are made so that the motion occurs entirely in the sagittal plane. Several embodiments of the registration process are disclosed herein below.

FIG. 2A is an illustration of a first movement for the registration process in accordance with an example embodiment. The first movement is a heel slide activity. The person 16 sits in a chair 13 and slides their foot 15 along the floor 14 in a straight line. The hip joint 2, knee joint 5, and ankle joint 8 rotate only in the flexion and extension directions. In one embodiment, foot 15 has to move a predetermined distance during the registration process using the first movement. In one embodiment, the first movement may be repeated more than one time to generate sufficient measurement data.

FIG. 2B is an illustration of a second movement for the registration process in accordance with an example embodiment. The second movement comprises lifting a foot of person 16. The second movement is also a seated activity as shown. An initial position of the second movement has person 16 with a foot on the floor and a toe coupling to a wall 17. Person 16 then lifts leg 108 while the toe continues to couple to wall 17. Note that the hip joint 2 has no translational motion while person 16 lifts leg 108. In one embodiment, foot 15 inclination relative to the wall remains approximately constant. In one embodiment, foot 15 has to move a predetermined distance during the registration process using the second movement. In one embodiment, the second movement may be repeated more than one time to generate sufficient measurement data.

FIG. 2C is an illustration of a third movement for the registration process in accordance with an example embodiment. The third movement is a standing position movement. The initial position for the third movement is for the patient 16 to have a back coupled to wall 17 and a heel of foot 15 coupled to wall 17 as shown. In the example, person 16 slides foot 15 away from wall 17 sliding foot 15 along floor 14 while maintaining the back of person 16 coupled to wall 17. In one embodiment, foot 15 has to move a predetermined distance during the registration process using the third movement. In one embodiment, the third movement may be repeated more than one time to generate sufficient measurement data.

FIG. 2D is an illustration of a fourth movement for the registration process in accordance with an example embodiment. The fourth movement is a standing position movement. The initial position of the fourth movement has the back of person 16 coupled to wall 17 with the heel of foot 15 on floor 14 and against wall 17. Person 16 then raises foot 15 above floor 14 with the heel of foot 15 coupled to wall 17. In the example, it is the ankle joint 8 that needs to move although foot 15 is disclosed as moving. In one embodiment, the tibia is the body segment with constrained movement as the end of the tibia with ankle joint 8 needs to remain in the same relative distance to the wall. In one embodiment, foot 15 has to move a predetermined distance during the registration process using the fourth movement. In one embodiment, the fourth movement may be repeated more than one time to generate sufficient measurement data.

FIG. 2E is an illustration of a fifth movement for the registration process in accordance with an example embodiment. The fifth movement is a standing position movement. The initial position of the fifth movement has the back of person 16 coupled to wall 17 with a heel of foot 15 coupled to wall 17 and foot 15 on floor 14. In the example, foot 14 is not the constrained moving segment. Foot 15 remains stationary while person 16 squats with the back coupled to the wall thereby moving hip joint 2 in a straight line as shown. In one embodiment, the center of hip joint 2 has to move a predetermined distance during the registration process using the fifth movement. In one embodiment, the fifth movement may be repeated more than one time to generate sufficient measurement data. Although, all the examples shown herein above are for a femur and a tibia of a leg, the movements can be adapted for other parts of the musculoskeletal for the registration process such as the elbow, shoulder, hip, ankle, wrist, hand, toes, or spine.

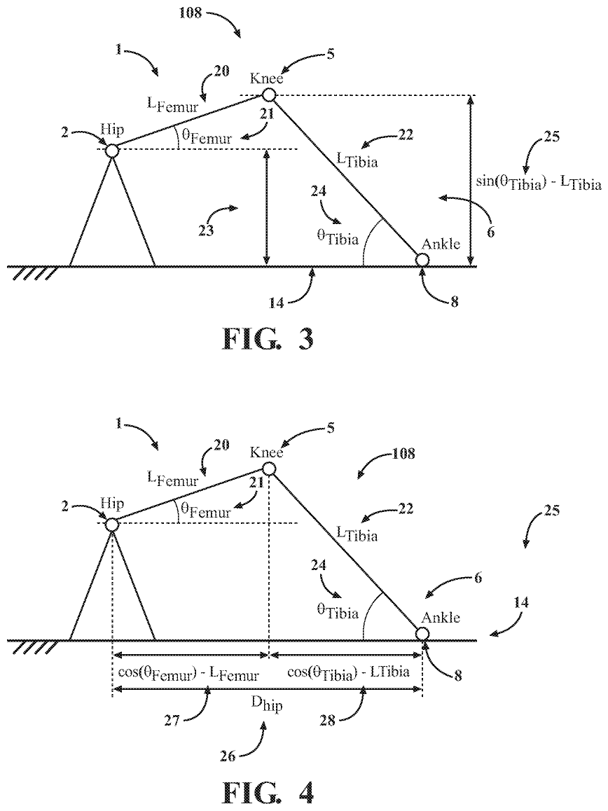

FIG. 3 is a geometric representation of the body segments as disclosed herein above for the first movement in accordance with an example embodiment. The first movement is in the sitting position and sliding the foot on the floor. In the example, the segments are femur 1 and tibia 6 of leg 108 of FIG. 2A will be referred to herein below. A geometric diagram of the body segments during the floor slide activity of FIG. 2A is illustrated in FIG. 3. In the example, L_{Femur} 20 and L_{Tibia} 22 are the lengths of the femur 1 and tibia 6. L_{Femur} 20 is a distance from the knee center of knee joint 5 to the hip center of hip joint 2. L_{Tibia} 22 is a distance from the knee center of knee joint 5 to the ankle center of ankle joint 8. θ_{Femur} 21 is the inclination of femur 1 relative to the floor 14, while 0 Tibia 24 is the inclination of the tibia 6 relative to the floor 14. Hₕᵢₚ 23 is the height of the hip joint 2 above floor 14. In one embodiment, hip joint 2 does not translate during the activity thereby fillip 23 will be constant and all poses of leg 108 during the floor slide known as the first movement will satisfy the equation 1 listed herein below.

Hₕᵢₚ=L_{Tibia} sin θ_{Tibia}−L_{Femur} sin θ_{Femur} Equation 1:

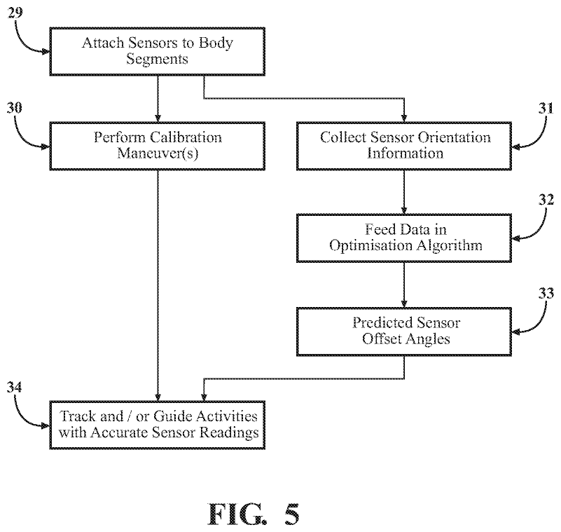

FIG. 4 is a geometric representation of the body segments as disclosed herein above the second movement in accordance with an example embodiment. The second movement is in the sitting position with the foot on the floor and a toe coupled to the wall. The foot is then raised from the wall with the toe maintaining coupling to the wall during the second movement. This is also called the wall slide activity as shown in FIG. 2B. Dₕᵢₚ is the distance from the wall 17 of FIG. 2B to the hip center of hip joint 2. Similar to the first movement, the distance from wall 17 of FIG. 2B to the hip joint 2 is assumed to be constant, and therefore all poses of leg 108 will satisfy the equation 2 shown herein below.

Dₕᵢₚ=L_{Femur} cos θ_{Femur}+L_{Tibia} cos θ_{Tibia} Equation 2:

Similar equations can be derived for other activities as shown in FIG. 3. The following algorithm can be extended to other movements for performing a registration process as such as the third, fourth, and fifth movements disclosed herein above. When these activities are performed, the true values of L_{Femur} 20, L_{Tibia} 22, θ_{Femur} 21, θ_{Tibia} 24, Hₕᵢₚ 23, and Dₕᵢₚ 26 are unknown. However, the equations can be manipulated to reduce the number of unknown quantities. First, both equation 1 and 2 are divided by L_{Femur} 20. Then, new constants Ci and Ci are defined, which are equal to Hₕᵢₚ and Dₕᵢₚ divided by L_{Femur} 20. Finally, L_{Tibia}/L_{Femur} can be is reduced to a constant L_{R}, which is the leg length ratio. Thus, a final form of the equations 1 and 2 after performing the mathematical manipulation above result in equations 3 and 4 shown herein below.

cos θ_{Femur}+L_{R} cos θ_{Tibia}=C₁ Equation 3:

−sin θ_{Femur}+−L_{R} sin θ_{Tibia}=C₂ Equation 4:

Since the placement of each device 9 and device 106 of FIG. 1 relative to the mechanical axes of leg 108 are unknown, θ_{Femur} 21 and θ_{Tibia} 24 are be represented as the sum of two quantities, where the first is the time varying sensor inclination angle readings, θ_{Femur, sensor} and θ_{Tibia, sensor}, and the second is a constant angle offset applied to the sensor reading to align it with the mechanical axis, δ_{F} 10 and δ_{T} 12 θ_{Femur} 21 and θ_{Tibia} 24 are now represented by the equations 5 and 6 shown herein below.

θ_{Femur}==θ_{F,sensor}(t)+δ_{F} Equation 5:

θ_{Tibia}=θ_{T,sensor}+δ_{T} Equation 6:

The equations for each movement now become equations 7 and 8 shown herein below.

cos(θ_{F,sensor}(t)+δ_{F})+L_{R} cos(θ_{T,sensor}(t)+δ_{T})=C₁ Equation 7:

−sin(θ_{F,sensor}(t)+δ_{T})+−L_{R} sin(θ_{T,sensor}(t)+δ_{T})=C₂ Equation 8:

It is assumed that C₁ and C₂ are constant for all values of θ_{F, sensor} and θ_{T sensor} during the registration process. Therefore, the variation, represented any number of ways, of C₁ and C₂ should be zero for each activity or movement disclosed herein. For example, the variance of both sides of the above equations will be zero as shown herein below.

var(cos(θ_{F,sensor}+δ_{F})+L_{R} COS(θ_{T,sensor}+δ_{T}))=var(C₁)=0 Equation 9:

var(sin(θ_{F,sensor}+δ_{F})+−L_{R} sin(θ_{T,sensor}+δ_{T}))=var(C₂)=0 Equation 10:

Equations 9 and 10 have three unknown variables. One way to estimate values for these variables is to use mathematical optimization. Mathematical optimization is a technique for determining values of variables that maximize or minimize a function. In the example, values for the two offsets and L_{R} would be determined such that the variance of Equations 9 and 10 are as close to zero as possible for all sensor readings in the registration movements.

The formal optimization problem is defined for a single activity as stated in equation 11 shown herein below.

minimize f(δ_{F},δ_{T},L_{R}) where f(δ_{F},δ_{T},L_{R})=var(cos(θ_{F,sensor}+δ_{F})+L_{R} COS(θ_{T,sensor}+δ_{T}))² Equation 11:

Equation 11 can be extended to multiple activities by adding additional terms to the cost function as shown in the equation 12 shown herein below.

f(δ_{F},δ_{T},L_{R})=var(cos(θ_{F1,sensor}+δ_{F})+L_{R} COS(θ_{T1,sensor}+δ_{T}))²+var(−sin(θ_{F2,sensor}+δ_{F})+−L_{R} sin(θ_{T2,sensor}+δ_{T}))² Equation 12:

In equation 12, the theta values labeled as 1 and 2 come from two different activities. The variance of the equations for each activity must be calculated separately since the distance from the reference plane to the fixed joint might not be the same for both motions. For instance, the height of hip joint 2 during heel slides (first movement) might not be the same as the distance from wall 17 to hip joint 2 during a wall slide (second movement). Note that the sensor angles need to be measured with respect to the plane that foot 15 slides along. When doing wall lifts, a cosine function is used instead of sine, which is equivalent to adding or subtracting 90 degrees to the inclination angle.

The optimization problem can be solved using a variety of optimization methods, including but not limited to gradient based solvers and random search algorithms.

Thus, the registration process using one or more movements is configured to calculate an alignment of devices 9 and 106 to the underlying anatomical structure. Devices 9 and 106 will each have an orientation sensor such as an IMU. This can be achieved for a number of joints. Whereas the previous explanation focused on knee joint 5, the logic can be applied to a number of other joints, including but not limited to the elbow, the ankle, and the shoulder.

While the algorithm is explained using 2 dimensional equations with the assumption of planar motion, it can be extended to three dimensions as well. This is particularly relevant when the devices are placed subcutaneous, directly attached to the bone as is the case for implantable devices. Referring back to knee joint 5, devices 9 and 106 can track the movement of the tibio-femoral joint along three axes, hence also quantifying the varus/valgus and internal/external rotation. For a three-dimensional problem, the sine and cosine from the above equations are replaced with rotation matrices calculated from the IMU angles in devices 9 and 106. The optimization problem is then formulated to minimize the variation of the movement of the end of the limb in two dimensions instead of one.

The model for the three-dimensional formulation is shown in Equation 13 herein below.

Equation 13: p_{Hip}^{ankle}=R_{F,Sensor}^{R}R_{Femur}^{F,ensor}l_{Femur}+R_{T,Sensor}^{R}R_{Tibia}^{T,sensor}l_{Tibia}

In equation 13, p_{Hip}^{ankle} is the vector from hip joint 2 to ankle joint 8, expressed in the room reference frame. R_{Femur}^{F,ensor} is the rotation matrix from femur 1 to device 9 coordinates, a function of δ̅_{F} 10. δ̅_{F} 10 is a sequence of rotations or a quaternion determined via optimization. R_{F,sensor}^{R} is the rotation matrix from the femur 1 device 9 to the room, determined from the uncorrected device 9 angles. l_{Femur} is the distance vector from hip joint 2 to knee joint 5, expressed in femur 1 coordinates. For simplicity, this vector can be assumed to be [0,L_{Fenmr},0]. R_{Tibia}^{T,Sensor} is the rotation matrix from tibia 6 to device 106, a function of δ̅_{T} 12. δ̅^{T} 12 is a sequence of rotations or a quaternion determined via optimization. R_{T,Sensor}^{R} is the rotation matrix from device 106 to the room coordinates, l_{Tibia} is the distance vector from knee joint 5 to ankle joint 8, expressed in the tibia 6 coordinates. For simplicity,/Tibia can be assumed to be [0,L_{Tibia},0].

For the optimization problem in three dimensions, ankle joint 8 is assumed to move in a straight line, therefore the variance of the ankle path in two dimensions can be minimized.

minimize f(δ̅_{F},δ̅_{T}) where f(δ̅_{F},δ̅_{T})=var(p_{Hip}^{Ankle}(t)·ĵ)²+var(p_{Hip}^{Ankle}(t)·\hat{k})² Equation 14:

In Equation 14, the position of ankle joint 8 for each time frame is calculated using equation 13. ĵ and \hat{k} are orthogonal unit vectors in the two constrained directions, represented in the room coordinate system. For simplicity, these unit vectors can be assumed to be parallel to two room coordinate axes.

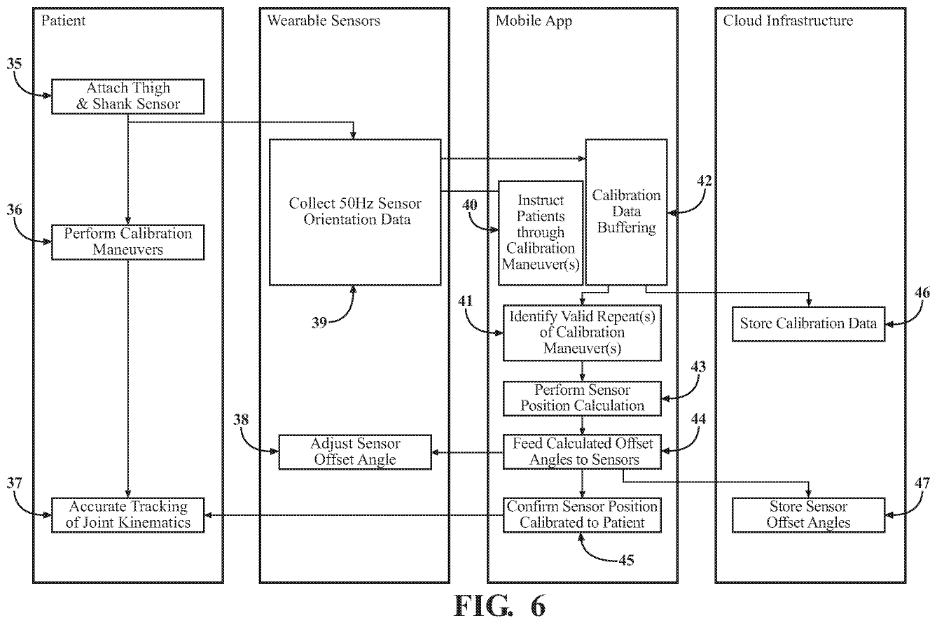

FIG. 5 is a flow chart of a registration process in accordance with an example embodiment. In a block 29, a first device and a second device having orientation sensors are coupled to segments of the musculoskeletal system. In the example, device 9 is coupled to a femur and device 106 is coupled to a tibia. Examples, of the first device and the second device can be a patch attached to skin, a surgical navigation system, or a sub-dermal implantable device. In block 30, a person to which the first and second devices couple then performs one of the calibration or registration maneuvers, for example a floor slide and/or a wall slide as disclosed herein above, with the instrumented limb. In a block 31, the first device and the second device transmit measurement data to a computer to process, analyze, and complete the registration process. In block 32, measurement data from the first and second devices during one or more movements is then coupled to an optimization algorithm in the computer. In a block 33, the computer calculates the offsets and leg length ratio to complete the registration process. In a block 34, the first and second device alignment is updated in the computer so that the first and second devices can be used to track or guide subsequent activities related to segments of the musculoskeletal system.

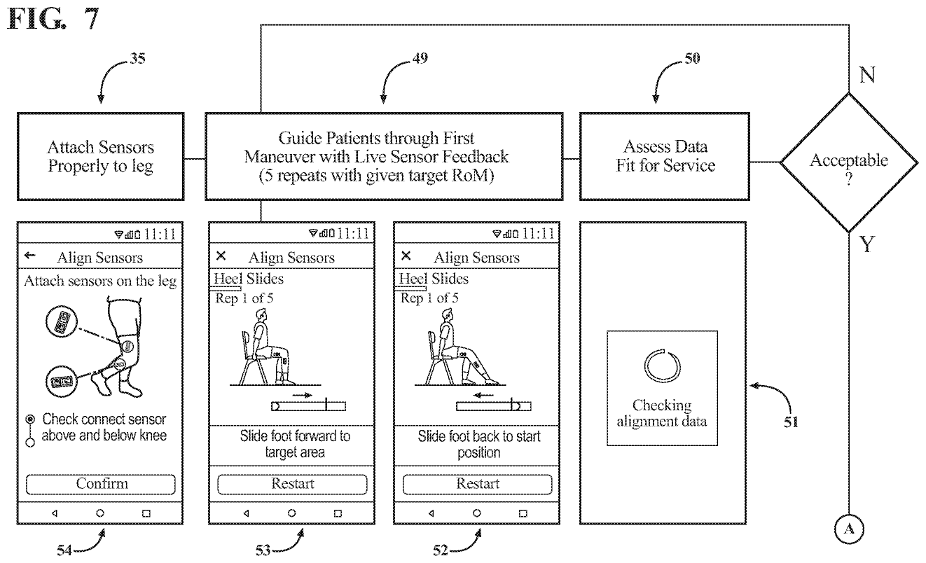

FIG. 6 block diagram showing an overview of a data flow for the knee example in accordance with an example embodiment. In a block 35, devices having at least one orientation sensor are coupled to segments of the musculoskeletal system. In the example, the segments can comprise an arm or a leg. In a block 36, a person performs a calibration or registration process while being given feedback by an application. The calibration or registration process generates measurement data from the devices during one or movements that is transmitted to a computer. In one embodiment, the computer is a hand held device, a tablet, a cell phone, a notebook computer, or other device that can run an application. In one embodiment, the computer is a tablet having a display that can be placed near the person performing the movement for viewing the feedback. In block 39, the measurement data from the calibration is passed to block 42 which is a calibration data buffering storage. In block 46, the calibration data is received from calibration data buffering storage and is sent to permanent storage in the cloud. In block 41 the measurement data is analyzed to identify if the measurement data received is valid (i.e. the correct maneuvers were performed). In block 43, if the data is valid, the calibration position calculation is performed. In one embodiment, the calibration positioning calculation is performed using cloud computing. In block 44, the calculated sensor offsets related to the devices are passed to the devices to determine adjusted sensor angles. In block 47 the sensor offsets related to the devices are also sent to cloud storage 47. In a block 45, a final confirmation of the calibration accuracy is performed. In a block 37, the resulting joint angle measurements are tracked for clinical use.

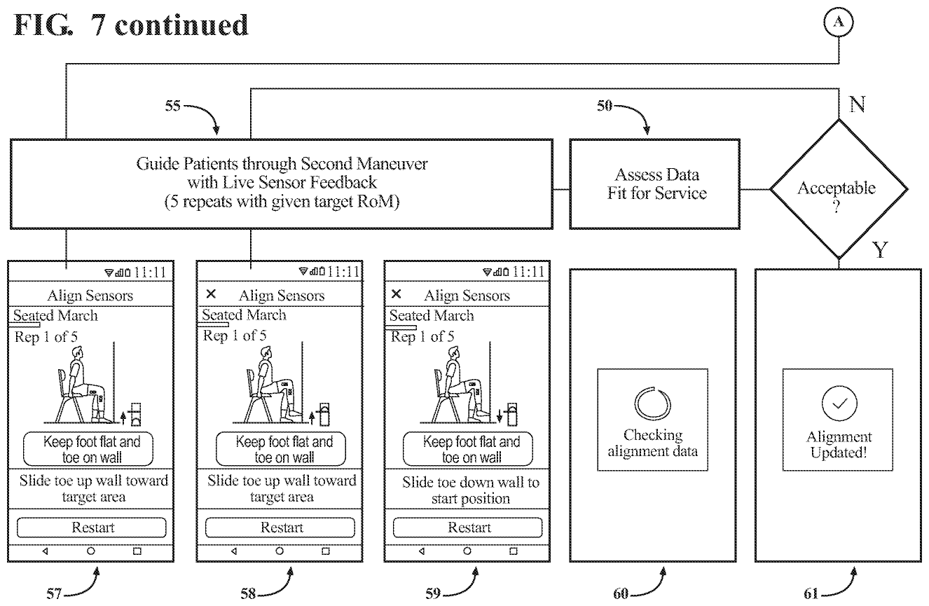

FIG. 7 is a block diagram showing one or more images of an application for performing the registration process on a computer in accordance with an example embodiment. In the example, the computer is a cell phone with a display. The application is installed on cell phone. The application is interactive with the user for providing feedback to the user in real-time. The block diagram gives a detailed view on a user interface on the abovementioned mobile application showing how the user walks through one or more calibration movements to perform a registration process. The example registration process is for a leg of the user. In the block 35, a first device is coupled to the femur and a second device is coupled to the tibia. In the example, the first device and the second device are patch devices coupled to the skin of the user. In block 54, the application displays areas to which the first device and the second device are coupled and can provide instructions.

In block 49, the application guides the patient or user through at least one movement. In one embodiment, the user is guided through a first movement and repeats the movement five times with a given target range of motion to collect sufficient measurement data for the registration process. In block 53, the user is sitting in a chair in the initial position with the foot on the floor as disclosed in FIG. 2A. Note that the movement bar shows the foot at the initial position. In block 52, the user is sliding the foot on the floor. The application will change the position of the leg in the display of the device running the application in real-time. The movement bar on the display also shows the distance moved as the user slides the foot along the floor in real-time. In the example, the movement bar indicates the distance the leg has moved on the floor and the direction of the movement in real-time. In one embodiment, it shows the change in tibial tilt angle, relative to gravity, in a planar motion. In one embodiment, the distance bar includes a predetermined distance indicator. For example, the display indicates when the foot has traversed the predetermined distance or showing the change in tibial tilt angle relative to gravity. Alternatively, the computer can provide other visual, audible, or haptic feedback to indicate that the predetermined distance has been traversed. In one embodiment, after reaching the predetermined distance the first movement can also include sliding the foot back to the initial position. Note that on the application the movement bar indicates that foot has traversed the predetermined distance indicator (e.g. the line on the movement bar) and that the foot is being moving back to the initial position (as indicated by the foot movement direction indicator arrow).

In block 50, the measurement data received from the one or more first movements by the user is being processed by the computer running the application. In general, an optimization algorithm is running in the background and processing the measurement data received from the first and second devices coupled to the segments of the musculoskeletal system. In block 51, the application indicates that the measurement data is being checked after at least one of the first movements is completed. If the measurement data is not acceptable the application directs the user to block 49 to repeat the first movement for collecting more measurement data. If the measurement data is acceptable for the registration process the application can move to block 55.

In block 55, the user is guided through a second maneuver with live sensor feedback. In one embodiment, the second maneuver is repeated five times with a given range of motion. In the example, the second movement as shown in FIG. 2B is performed by the user of the application. In block 57, the user is shown in the initial position of the second movement. In the example, the user is sitting in a chair with the foot on the floor and the toe against a wall. The display of the device running the application shows an image of the user in the initial position. The display further shows a movement bar with the foot in the initial position. Note that movement bar is vertical as the foot will be raised and lowered with the toe touching the wall. In block 58, the user begins the second movement. The display shows the foot being raised. The movement bar indicates the distance or the angular change of the thigh sensor of the second movement and the direction of the second movement. The display also indicates a predetermined distance that must be traversed. In the example, the predetermined distance is indicated by a line through the movement bar. In the example, the display indicates that the foot is raised above the predetermined distance. The computer can provide visual, audible, or haptic feedback indicating that the predetermined distances has been exceeded. In block 59, the display shows the foot being lowered while maintaining the toe against the wall. Note that the arrow indicates the direction of the movement as the foot is brought back to the initial position. In block 50, the measurement data from the first and second devices is assessed. In block 60, the application indicates that the measurement data is being checked after at least one of the second movements is completed. If the measurement data is not acceptable the application directs the user to block 55 to repeat the second movement for collecting more measurement data. If the measurement data is acceptable for the registration process the application can move to block 61. In block 61, the measurement data has been found acceptable and the user is notified that the first movement and the second movement measurement data has been received and processed. The optimization algorithm has been running in the background using the measurement data to complete the registration process. Once completed, the alignment of the first and second devices is updated. Thus, movements of the first and second segments of the musculoskeletal system can be accurately tracked after the registration process to support monitoring kinematics of the musculoskeletal system.

The following kinematics tracking system will be discussed herein below using components from FIGS. 1-7. The components disclosed will be from at least one of FIGS. 1-7. The kinematics tracking system comprises a device 9, a device 106, and a computer 102. Device 9 is configured to measure orientation. Device 106 is configured to measure orientation. Device 9 and device 106 are respectively couple to a first segment and a second segment of the musculoskeletal system of a person. Computer 102 is configured to receive measurement data from device 9 and device 106. Computer 102 includes at least one application configured to direct a person to perform a registration process. The application can include visual, audible, or haptic queues to support the person performing one or more movements. The registration process determines a zero level by kinematic axes coupling rotational joints to the first and second segments. The application on the computer is configured to direct the person through at least one movement of the first or second segment. The at least one movement comprises the first segment configured to rotate and the second segment is configured to move in a straight line while remaining rotationally free. In the example, the first movement of the leg 108, hip joint 2 is configured to rotate and ankle joint 8 is configured to move in the straight line. The measurement data collected during the at least one movement is used to determine a rotation of 6F and 6T of the device 9 and device 106 such that the zero level corresponds to a kinematic axis coupling the rotational joints with a shared joint coupled between the first and second segments. In the example, the rotational joints are hip joint 2 and ankle joint 8. The shared joint is knee joint 5.

In one embodiment, device 9 and device 106 are configured to couple to skin respectively coupling to the first segment and the second segment of the musculoskeletal system. Device 9 and device 106 each have at least one inertial measurement unit (IMU) configured to measure orientation. In one embodiment, computer 102, device 9, and device 106 comprises a navigation system for monitoring orientation of the musculoskeletal system. In one embodiment, device 9 is an implanted device coupled to the femur of the leg. Device 106 is an implanted device coupled to the tibia of the leg. The implanted devices 9 and 106 coupled to bone are sub-dermal.

Computer 102 includes a display 104. The display is configured to indicate a position of the first segment and the second segment. In the example, the application displays leg 108 during the one or more movements. The first segment of leg 108 is the femur and the second segment of leg 108 is the tibia. The display 104 is configured to indicate movement of the femur and the tibia. In one embodiment, the femur and the tibia is displayed as leg 108 having a thigh and shank. The movement of leg 108 on the display is shown in real-time. The application on computer 102 further includes a movement bar configured to show movement related to the femur or the tibia. The person performing the at least one movement will see on the display movement related to the femur or tibia in real-time as the at least one movement is performed. The display will indicate when the at least one movement has traveled a predetermined distance or angle. Computer 102 is configured to provide visual, audible, or haptic feedback related to the at least one movement. In one embodiment, the at least one movement can be repeated until sufficient measurement data has been collected.

The rotational joints comprises a first segment end coupling to a joint that provides rotational freedom and a second segment end coupling to a joint that provides rotational freedom. In the example, the rotational joint coupled to the first segment is hip joint 2. The rotation joint coupled to the second segment is ankle joint 8. The shared joint coupling the first segment to the second segment is knee joint 5. The first segment is the femur of the leg and the second segment is the tibia of the leg. In one embodiment, the hip joint 2 is configured to have no translational movement during the movement of the leg used for the registration process. The computer is configured to receive the measurement data from device 9 and device 106 and calculate the rotation of 6F and 6T of device 9 and device 106 such that the zero level corresponds to a kinematic axis coupling the rotation joints with the shared joint.

In one embodiment, the at least one movement for the registration process comprises the person sitting in a chair and sliding a heel of a leg along a floor in a straight line as disclosed in FIG. 2A. In one embodiment, the at least one movement for the registration process comprises the person sitting in the chair and the person lifting the leg with a toe touching a wall as disclosed in FIG. 2B. In one embodiment, the at least one movement for the registration process comprises a back of the person coupled to a wall and sliding a heel of a leg from the wall in a straight line away from the wall as disclosed in FIG. 2C. In one embodiment, the at least one movement comprises the back of the person coupled to the wall and sliding the heel from the floor upward in a straight line along the wall as shown in FIG. 2D.

The kinematics tracking system comprises a device 9, a device 106, and a computer 102. Device 9 is configured to measure motion, position, or orientation. Device 9 is configured to couple to the femur of the leg of a person. Device 106 is configured to measure motion, position, or orientation. Device 106 is configured to couple to the tibia of the leg of the person. Computer 102 is configured to receive measurement data from device 9 and device 106. Computer 102 includes an application configured to direct the person to perform a registration process comprising a movement. The femur is configured to rotate and the tibia is configured to move in a straight line while remaining rotationally free in the movement. Computer 102 reports on a display 104 when tibia has moved a predetermined distance or angle.

Display 102 indicates a position of the femur (thigh) and tibia (shank) of the leg 108. Display 102 is configured to indicate movement of the femur (thigh) and the tibia (shank) of the leg 108 in real-time. The kinematics tracking system is configured to provide audible, visual, or haptic feedback to the person to support the registration process. The application run on computer 102 is configured to support the registration process using the movement of the leg 108. In one embodiment, the movement comprises the person sitting in a chair sliding a heel of the leg 108 along a floor in a straight line as shown in FIG. 2A. In one embodiment, the movement comprises the person sitting in the chair and the person lifting the leg 108 with a toe touching a wall as shown in FIG. 2B. In one embodiment, the movement comprises a back of the patient coupled to a wall and sliding a heel of the leg 108 from the wall in a straight line away from the wall as shown in FIG. 2C. In one embodiment, the movement comprises the back of the patient coupled to the wall and sliding the heel from the floor upward in a straight line along the wall as shown in FIG. 2D. A depiction of the person is on the display 104. The display 104 will show the movement of the person and more specifically leg 108 in real-time. Display 104 further includes a movement bar configured to show movement related to the second segment in real-time. The movement bar is shown in blocks 52, 53, 57, 58, and 59 related to the application that is on the computer. Display 104 indicates when the at least on movement has traveled a predetermined distance such that the movement can be repeated or stopped. In blocks 52, 53, 57, 58, and 59 the predetermined distance is indicated by a line. In one embodiment, the movement must exceed the line indicating the predetermined distance of the foot movement.

In the example, the femur and the tibia of leg 108 are adjacent. The zero level corresponds to a static calibration pose of the femur and the tibia. The zero level is determined by kinematic axes coupling rotational joints to the femur and the tibia. The rotational joint of the femur is hip joint 2. The rotational joint of the tibia is ankle joint 8. The shared joint is knee joint 5. The application on computer 102 is configured to direct the person through the movement of the femur and the tibia to determine a rotation of Sf and St as shown in FIG. 1 of device 9 and device 106 such that the zero level corresponds to the static calibration pose. The end of the femur is positioned to prevent movement of hip joint 2 in the movement. The end of the tibia is moved in a straight line. The femur does not have translation movement during the movement of the calibration process.

Just as each person is different, each device installation is different having many different variations. Having measured data and using the measurement data for monitoring movement will greatly increase the consistency of the implant procedure thereby reducing rework and maximizing the life of the implants. In at least one exemplary embodiment, the measured data can be collected to a database where it can be stored and analyzed. For example, once a relevant sample of the measured data is collected, it can be used to define optimal initial measured settings, geometries, and alignments for maximizing the life and usability of an implanted orthopedic device.

While the present invention has been described with reference to particular embodiments, those skilled in the art will recognize that many changes may be made thereto without departing from the spirit and scope of the present invention. Each of these embodiments and obvious variations thereof is contemplated as falling within the spirit and scope of the invention.

1. A kinematics tracking system comprising:
a first device configured to measure a first orientation wherein the first device is configured to be coupled to a first segment of a musculoskeletal system of a person;
a second device configured to measure a second orientation wherein the second device is configured to be coupled to a second segment of the musculoskeletal system of the person; and
a computer configured to receive measurement data from the first device and the second device wherein the computer includes an application configured to direct the person to perform a registration process, wherein a zero level is determined by kinematic axes coupling rotational joints to the first and second segments, wherein at least one movement comprises the first segment configured to rotate and the second segment is configured to move in a straight line while remaining rotationally free, wherein the measurement data collected during the at least one movement is used to determine a rotation of δ_{F} and δ_{T} of the first device and the second device such that the zero level corresponds to a kinematic axis connecting the rotational joints with a shared joint coupled between the first and second segments.

2. The kinematics tracking system of claim 1, wherein the rotational joints comprises a first segment end coupling to a first joint that provides rotational freedom and a second segment end coupling to a second joint that provides rotational freedom, wherein an end of the first segment end is configured to have no translational movement, and wherein the computer is configured to receive the measurement data and calculate the rotation of δ_{F} and δ_{T} of the first device and the second device such that the zero level corresponds to the kinematic axis coupling the rotational joints with the shared joint coupled between the first and second segments.

3. The kinematics tracking system of claim 1, wherein the first device is configured to couple to skin of the person, wherein the second device is configured to couple to the skin, wherein the first device includes at least one inertial measurement unit (IMU) configured to measure the first orientation, and wherein the second device includes at least one IMU configured to measure the second orientation.

4. The kinematics tracking system of claim 1 further comprising a navigation system for monitoring a pose of the first device and a pose of the second device.

5. The kinematics tracking system of claim 1, wherein the first device is a first implanted device configured to be coupled to the first segment, wherein the second device is a second implanted device configured to be coupled to the second segment, and wherein the first and second devices are subdermal.

6. The kinematics tracking system of claim 1, wherein the computer includes a display, wherein the display indicates a position of the first segment and the second segment, and wherein the display is configured to indicate movement of the first segment and the second segment in real-time.

7. The kinematics tracking system of claim 6, wherein the display includes a movement bar configured to show movement related to the second segment, wherein the at least one movement is displayed in real-time as the person performs the at least one movement, and wherein the display indicates when the at least one movement has traveled a predetermined distance or angle.

8. The kinematics tracking system of claim 7, wherein the computer is configured to provide haptic, visual, or audible feedback related to the at least one movement.

9. The kinematics tracking system of claim 8, wherein the at least one movement comprises the person sitting in a chair and sliding a heel of a leg along a floor in a straight line or wherein the at least one movement comprises the person sitting in the chair and the person lifting the leg with a toe touching a wall.

10. The kinematics tracking system of claim 8, wherein the at least one movement comprises a back of the person coupled to a wall and sliding a heel of a leg from the wall in a straight line away from the wall, or wherein the at least one movement comprises the back of the person coupled to the wall and sliding the heel from a floor upward in a straight line along the wall.

11. A kinematics tracking system comprising:
a first device configured to measure a first motion, a first position, or a first orientation wherein the first device is configured to be coupled to a first segment of a musculoskeletal system of a person;
a second device configured to measure a second motion, a second position, or a second orientation wherein the second device is configured to be coupled to a second segment of the musculoskeletal system of the person; and
a computer configured to receive measurement data from the first device and the second device wherein the computer includes an application configured to direct the person to perform a registration process comprising a movement, wherein the first segment is configured to rotate and wherein the second segment is configured to move in a straight line while remaining rotationally free, wherein the computer reports on a display when the second segment has moved a predetermined distance or angle.

12. The kinematics tracking system of claim 11, wherein the display indicates the first position of the first segment and the second position of the second segment, wherein the display is configured to indicate movement of the first segment and the second segment in real-time, and wherein the kinematics tracking system is configured to provide audible, visual, or haptic feedback to the person to support the registration process.

13. The kinematics tracking system of claim 12, wherein the application is configured to support the registration process using the movement, wherein the movement comprises the person sitting in a chair sliding a heel of a leg along a floor in a straight line or wherein the movement comprises the person sitting in the chair and the person lifting the leg with a toe touching a wall.

14. The kinematics tracking system of claim 12, wherein the application is configured to support the registration process using the movement, wherein the movement comprises a back of the person coupled to a wall and sliding a heel of a leg from the wall in a straight line away from the wall, or wherein the movement comprises the back of the person coupled to the wall and sliding the heel from a floor upward in a straight line along the wall.

15. The kinematics tracking system of claim 11, wherein the display further includes a movement bar configured to show movement related to the second segment in real-time, wherein the display indicates when the movement has traveled the predetermined distance such that the movement can be repeated or stopped.

16. The kinematics tracking system of claim 1, wherein the first and second segments are adjacent, wherein a zero level corresponds to a static calibration pose of the first and second segments, wherein the zero level is determined by kinematic axes coupling rotational joints to the first and second segments, wherein the application on the computer is configured to direct the person through the movement of the first or second segments to determine a rotation of δ_{F} and δ_{T} of the first device and the second device such that the zero level corresponds to the static calibration pose, wherein an end of the first segment is positioned to prevent movement of the end, and wherein an end of the second segment moves in a straight line.

17. The kinematics tracking system of claim 16, wherein the first segment does not have translational movement during the movement of the calibration process.

18. The kinematics tracking system of claim 17, wherein the first device is configured to couple to skin of the person, wherein the second device is configured to couple to the skin, wherein the first device includes an inertial measurement unit (IMU) configured to measure the first orientation, and wherein the second device includes an IMU configured to measure second orientation.

19. The kinematics tracking system of claim 18, further comprising a navigation system for monitoring a pose of the first device and a pose of the second device.

20. The kinematics tracking system of claim 19, wherein the first device is a first implanted device coupled to the first segment, wherein the second device is a second implanted device coupled to the second segment, and wherein the first and second devices are subdermal.
